(19) United States
(12) Patent Application Publication
(10) Pub. No.: US 2011/0208090 A1
(43) Pub. Date: Aug. 25, 2011

(54) Spring Loaded Biopsy Device

(76) Inventor: Shailendra K. Parihar, Mason, OH (US)

(21) Appl. No.: 12/709,624

(22) Filed: Feb. 22, 2010

(51) Int. Cl. A61B 10/02

(52) U.S. Cl. 600/568

(57) ABSTRACT

A biopsy device comprises a body, a needle, a cutter, and a motor. The cutter is translatable within the needle to sever tissue protruding through a lateral aperture formed in the needle. A cutter actuation mechanism comprises a split nut, a cutter retraction nut, and a resilient member. The resilient member is configured to bias the cutter distally. The motor is operable to rotate the split nut, which in turn translates the cutter retraction nut proximally in order to translate the cutter proximally and compress the resilient member. Once the cutter reaches a proximal position, segments forming the split nut separate to disengage the cutter retraction nut, thereby allowing the resilient member to translate the cutter distally. In addition, the motor rotates the cutter as the cutter translates distally. The motor may selectively engage either the split nut or the cutter for rotation.

### BACKGROUND

Biopsy samples have been obtained in a variety of ways in various medical procedures using a variety of devices. Biopsy devices may be used under stereotactic guidance, ultrasound guidance, MRI guidance, PEM guidance, BSGI guidance, or otherwise. For instance, some biopsy devices may be fully operable by a user using a single hand, and with a single insertion, to capture one or more biopsy samples from a patient. In addition, some biopsy devices may be tethered to a vacuum module and/or control module, such as for communication of fluids (e.g., pressurized air, saline, atmospheric air, vacuum, etc.), for communication of power, and/or for communication of commands and the like. Other biopsy devices may be fully or at least partially operable without being tethered or otherwise connected with another device.

Merely exemplary biopsy devices are disclosed in U.S. Pat. No. 5,526,822, entitled “Method and Apparatus for Automated Biopsy and Collection of Soft Tissue,” issued Jun. 18, 1996; U.S. Pat. No. 6,086,544, entitled “Control Apparatus for an Automated Surgical Biopsy Device,” issued Jul. 11, 2000; U.S. Pub. No. 2003/0109803, entitled “MRI Compatible Surgical Biopsy Device,” published Jun. 12, 2003; U.S. Pub. No. 2006/0074345, entitled “Biopsy Apparatus and Method,” published Apr. 6, 2006; U.S. Pub. No. 2007/0118048, entitled “Remote Thumbwheel for a Surgical Biopsy Device,” published May 24, 2007; U.S. Pub. No. 2008/0214955, entitled “Presentation of Biopsy Sample by Biopsy Device,” published Sep. 4, 2008; U.S. Pub. No. 2009/0171242, entitled “Clutch and Valving System for Tetherless Biopsy Device,” published Jul. 2, 2009; U.S. Non-Provisional patent application Ser. No. 12/335,578, entitled “Hand Actuated Tetherless Biopsy Device with Pistol Grip,” filed Dec. 16, 2008; U.S. Non-Provisional patent application Ser. No. 12/337,942, entitled “Biopsy Device with Central Thumbwheel,” filed Dec. 18, 2008; and U.S. Non-Provisional patent application Ser. No. 12/483,305, entitled “Tetherless Biopsy Device with Reusable Portion,” filed Jun. 12, 2009. The disclosure of each of the above-cited U.S. patents, U.S. Patent Application Publications, and U.S. Non-Provisional Patent Applications is incorporated by reference herein.

While several systems and methods have been made and used for obtaining a biopsy sample, it is believed that no one prior to the inventors has made or used the invention described in the appended claims.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements and in which:

FIG. 1 depicts a perspective view of an exemplary biopsy device;

FIG. 2 depicts a partial cross-sectional view of the biopsy device of FIG. 1, showing cutter actuation components, with the cutter in an initial, distal position;

FIG. 3 depicts a partial view of the needle of the biopsy device of FIG. 1, with the needle shown in cross section and with the cutter in the initial, distal position;

FIG. 4 depicts a partial cross-sectional view of the biopsy device of FIG. 1, showing cutter actuation components, with the cutter retracted to a proximal position;

FIG. 5 depicts a partial view of the needle of the biopsy device of FIG. 1, with the needle shown in cross section and with the cutter in an intermediate position during retraction;

FIG. 6 depicts a partial view of the needle of the biopsy device of FIG. 1, with the needle shown in cross section and with the cutter in the retracted, proximal position;

FIG. 7 depicts a partial cross-sectional view of the biopsy device of FIG. 1, showing cutter actuation components, with the cutter fired to the distal position, and with the split nut in a separated configuration;

FIG. 8 depicts a partial view of the needle of the biopsy device of FIG. 1, with the needle shown in cross section and with the cutter in the fired, distal position;

FIG. 9 depicts an end view of a split nut and cutter retraction nut of the biopsy device of FIG. 1, viewed from the distal side, with the split nut in a non-separated configuration;

FIG. 10 depicts an end view of the split nut and cutter retraction nut of FIG. 9, again viewed from the distal side, with the split nut in a separated configuration; and

FIG. 11 depicts an exploded perspective view of cutter actuation components of the biopsy device of FIG. 1.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

Overview

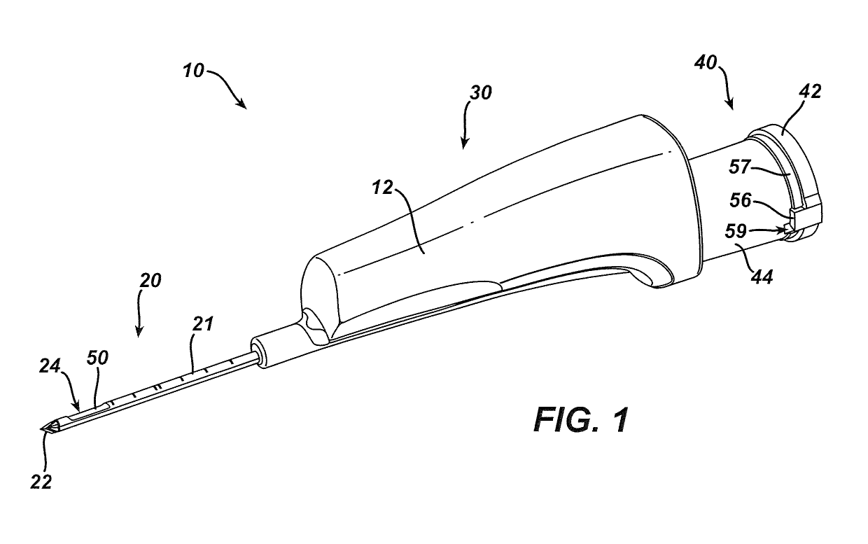

As shown in FIG. 1, an exemplary biopsy device (10) comprises a needle (20), a body (30), and a tissue sample holder (40). In particular, needle (20) extends distally from the distal portion of body (30), while tissue sample holder (40) extends proximally from the proximal portion of body (30). Body (30) is sized and configured such that biopsy device (10) may be operated by a single hand of a user. In particular, and as described in greater detail below, a user may grasp body (30) with a single hand, insert needle (20) into a patient's breast, and collect one or a plurality of tissue samples from within the patient's breast, all with just using a single hand Alternatively, a user may grasp body (30) with more than one hand and/or with any desired assistance. In some settings, the user may capture a plurality of tissue samples with just a single insertion of needle (20) in the patient's breast. Such tissue samples may be pneumatically deposited in tissue sample holder (40), as described in greater detail below, then retrieved from tissue sample holder (40) for analysis.

Body (30) of the present example comprises a housing (12). In some version, body (30) is formed in at least two pieces, comprising a probe portion and a holster portion. For instance, in some such versions, the probe portion may be separable from the holster portion. Furthermore, the probe portion may be provided as a disposable component while the holster portion may be provided as a reusable portion. By way of example only, such a probe and holster configuration may be provided in accordance with the teachings of U.S. Non-Provisional patent application Ser. No. 12/483,305, entitled “Tetherless Biopsy Device with Reusable Portion,” filed Jun. 12, 2009. Alternatively, any other suitable probe and holster configuration may be used. It should also be understood that body (30) may be configured such that it does not have a probe portion and holster portion. Various other suitable ways in which body (30) may be configured will be apparent to those of ordinary skill in the art in view of the teachings herein.

While examples described herein refer to the acquisition of biopsy samples from a patient's breast, it should be understood that biopsy device (10) may be used in a variety of other procedures for a variety of other purposes and in a variety of other parts of a patient's anatomy.

Exemplary Needle

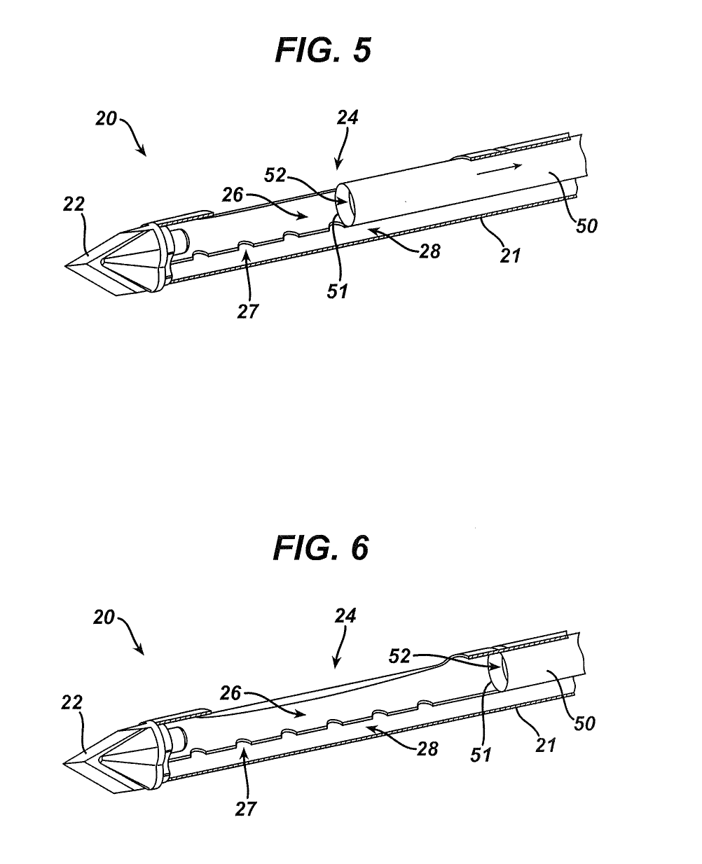

As shown in FIGS. 1-8 (among others), needle (20) of the present example comprises a cannula (21) with a tissue piercing tip (22), a lateral aperture (24), a first lumen (26), and a second lumen (28). Tissue piercing tip (22) is configured to pierce and penetrate tissue, without requiring a high amount of force, and without requiring an opening to be pre-formed in the tissue prior to insertion of tip (22). Alternatively, tip (22) may be blunt (e.g., rounded, flat, etc.) if desired. A cutter (50) is disposed in first lumen (26), and is operable to rotate and translate within first lumen (26) as will be described in greater detail below. Lateral aperture (24) is located proximal to tip (22), is in fluid communication with first lumen (26), and is configured to receive tissue when needle (20) is inserted in a breast and when a cutter (50) is retracted as will also be described in greater detail below. A plurality of openings (27) provide fluid communication between first and second lumens (26, 28). A plurality of external openings (not shown) may also be formed in needle (20), and may be in fluid communication with second lumen (28). For instance, such external openings may be configured in accordance with the teachings of U.S. Pub. No. 2007/0032742, entitled “Biopsy Device with Vacuum Assisted Bleeding Control,” published Feb. 8, 2007, the disclosure of which is incorporated by reference herein. Cutter (50) may also include one or more side openings. Of course, as with other components described herein, such external openings in needle (20) and cutter (50) are merely optional.

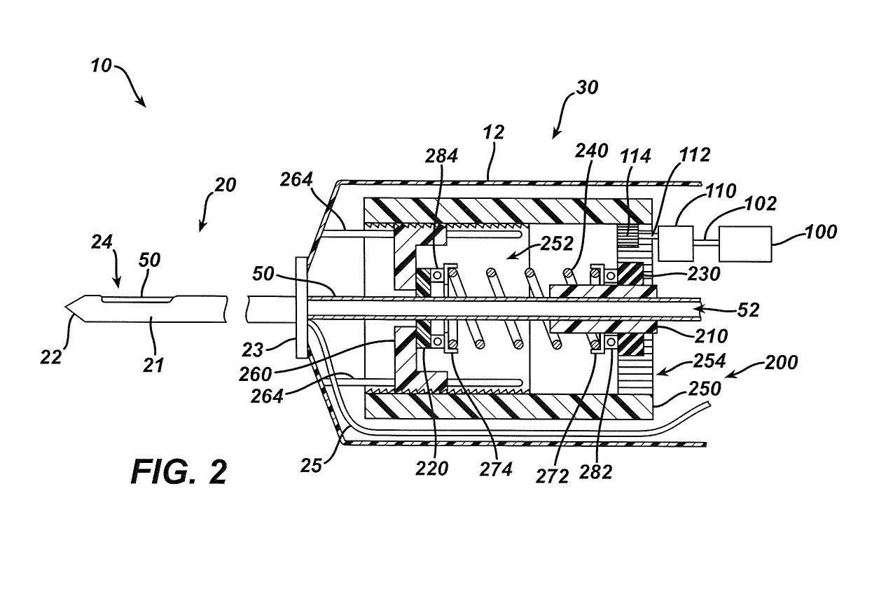

Needle (20) of the present example further comprises a hub (23), as shown in FIGS. 2, 4, and 7. Hub (23) may be formed of plastic that is overmolded about needle (20) or otherwise secured to needle (20), such that hub (23) is unitarily secured to needle (20). Alternatively, hub (23) may be formed of any other suitable material through any suitable process and may have any other suitable relationship with needle (20). Hub (23) of the present example is coupled with a vacuum conduit (25), and is operable to communicate a vacuum (or atmospheric air, saline, pressurized fluid, etc.) from vacuum conduit (25) to second lumen (28). Vacuum conduit (25) may be coupled with a variety of sources, including but not limited to a vacuum source that is external to biopsy device (10) or a vacuum source that is located within body (30). For instance, such an internal vacuum source may be provided in accordance with the teachings of U.S. Non-Provisional patent application Ser. No. 12/483,305, entitled “Tetherless Biopsy Device with Reusable Portion,” filed Jun. 12, 2009, the disclosure of which is incorporated by reference herein. In addition or in the alternative, vacuum conduit (25) may be coupled with an external vacuum control module in accordance with the teachings of U.S. Pub. No. 2008/0214955, entitled “Presentation of Biopsy Sample by Biopsy Device,” published Sep. 4, 2008, the disclosure of which is incorporated by reference herein. Still other suitable fluid sources that vacuum conduit (25) may be coupled with will be apparent to those of ordinary skill in the art in view of the teachings herein. Of course, any suitable type of valve(s) and/or switching mechanism(s) may also be coupled with vacuum conduit (25).

In some merely illustrative alternative versions, hub (23) is configured to provide a shuttle valve mechanism to selectively vent/seal second lumen (28), as taught in U.S. Non-Provisional patent application Ser. No. 12/483,305, entitled “Tetherless Biopsy Device with Reusable Portion,” filed Jun. 12, 2009.

It should be understood that, as with other components described herein, needle (20) may be varied, modified, substituted, or supplemented in a variety of ways; and that needle (20) may have a variety of alternative features, components, configurations, and functionalities. By way of example only, needle (20) may simply lack second lumen (28) altogether in some versions, such that first lumen (26) is the only lumen defined by needle (20). As another merely exemplary alternative, biopsy device (10) may be configured such that needle (20) may be fired distally relative to body (30), such as to assist in penetration of tip (22) in tissue. Such firing may be provided by one or more actuators (e.g., solenoid, pneumatic cylinder/piston, etc.), by one or more springs, or in any other suitable fashion; and may be activated by a push button or other feature of body (30). Other suitable alternative versions, features, components, configurations, and functionalities of needle (20) will be apparent to those of ordinary skill in the art in view of the teachings herein. Similarly, other suitable modifications to other components of biopsy device (10) that may be made in accordance with variations of needle (20) will be apparent to those of ordinary skill in the art in view of the teachings herein.

Exemplary Body

As noted above, body (30) of the present example comprises a housing (12), which may be provided in a plurality of assembled pieces if desired. A motor (100), gearbox (110), and cutter actuation mechanism (200) are provided within housing (12). Motor (100) of the present example comprises a conventional DC motor, though it should be understood that any other suitable type of motor may be used. By way of example only, motor (100) may comprise a pneumatic motor (e.g., having an impeller, etc.) that is powered by pressurized air, a pneumatic linear actuator, an electromechanical linear actuator, a piezoelectric motor (e.g., for use in MRI settings), or a variety of other types of movement-inducing devices. Motor (100) may be powered in a variety of ways. For instance, a rechargeable or non-rechargeable battery (not shown) may be provided within housing (12) to power motor (100). As another merely illustrative example, motor (100) may receive power from a power source that is external to body (30), such that motor (100) receives power via a cable. Various other suitable ways in which motor (100) may be powered will be apparent to those of ordinary skill in the art in view of the teachings herein.

Motor (100) is operable to rotate drive shaft (102), which extends distally from motor (100). Drive shaft (102) is in communication with gearbox (110) to provide a rotary input into gearbox (110). While drive shaft (102) is shown as extending directly from motor (100) into gearbox (110), it should be understood that a variety of other components may be coupled between motor (100) and gearbox (110), including but not limited to various gears, a clutch, etc. Gearbox (110) includes an output shaft (112) having a drive gear (114) secured thereto. As will be described in greater detail below, drive gear (114) is operable to selectively activate cutter actuation mechanism (200). Gearbox (110) may comprise a planetary gearbox, and may be configured to provide speed reduction. Various suitable configurations for gearbox (110) will be apparent to those of ordinary skill in the art in view of the teachings herein.

It should be understood that, as with other components described herein, body (30) may be varied, modified, substituted, or supplemented in a variety of ways; and that body (30) may have a variety of alternative features, components, configurations, and functionalities. As noted elsewhere herein, body (30) may include a vacuum pump. Such a vacuum pump may also be selectively activated by motor (100). Other suitable alternative versions, features, components, configurations, and functionalities of body (30) will be apparent to those of ordinary skill in the art in view of the teachings herein.

Exemplary Tissue Sample Holder

As shown in FIG. 1, tissue sample holder (40) of the present example comprises a cap (42) and an outer cup (44). A filter tray (not shown) is provided within outer cup (44). Cup (44) is secured to body (30) in the present example. Such engagement may be provided in any suitable fashion. Outer cup (44) of the present example is substantially transparent, allowing the user to view tissue samples on the filter tray, though outer cup (44) may have any other suitable properties if desired.

The hollow interior of outer cup (44) is in fluid communication with cutter lumen (52) and with a vacuum source (not shown) in the present example. Such a vacuum source may be within body (30) or external to body (30). By way of example only, vacuum may be provided to outer cup (44), and such a vacuum may be further communicated to cutter lumen (52), in accordance with the teachings of U.S. Non-Provisional patent application Ser. No. 12/483,305, entitled “Tetherless Biopsy Device with Reusable Portion,” filed Jun. 12, 2009, the disclosure of which is incorporated by reference herein. As another merely illustrative example, vacuum may be provided to outer cup (44) from an external vacuum source in accordance with the teachings of U.S. Pub. No. 2008/0214955, entitled “Presentation of Biopsy Sample by Biopsy Device,” published Sep. 4, 2008, the disclosure of which is incorporated by reference herein. Various other suitable ways in which vacuum may be provided to outer cup (44) will be apparent to those of ordinary skill in the art in view of the teachings herein. It should also be understood that outer cup (44) may receive vacuum from the same vacuum source as vacuum conduit (25). Biopsy device (10) may further include one or more valves (e.g., shuttle valve, electromechanical solenoid valve, etc.) to selectively regulate communication of a vacuum and/or other fluids to outer cup (44) and/or vacuum conduit (25), regardless of whether outer cup (44) and vacuum conduit (25) are coupled with a common source of vacuum or other source of fluid.

Cap (42) is removably coupled with outer cup (44) in the present example. A pair of latches (56) provide selective engagement between cap (42) and outer cup (44). In particular, latches (56) engage a lip (57) of outer cup (44). Lip (57) has gaps (59) permitting passage of latches (56), such that a user may secure cap (42) to outer cup (44) by aligning latches (56) with gaps (59), pushing cap (42) onto outer cup (44), then rotating cap (42) past gaps (59) to engage latches (56) with lip (57). Alternatively, cap (42) may be secured to outer cup (44) in any other suitable fashion. An o-ring (not shown) provides a seal when cap (42) is engaged with outer cup (44). A vacuum may thus be maintained within outer cup (44) when cap (42) is secured to outer cup (44). In operation, a user may remove cap (42) to access tissue samples that have gathered on a filter tray (not shown) within outer cup (44) during a biopsy process. In the present example, cap (42) is removed by rotating cap (42) to align latches (56) with gaps (59), then pulling cap (42) off. Of course, cap (42) may be removed from outer cup (44) in any other suitable fashion.

Tissue sample holder (40) of the present example is configured to hold at least ten tissue samples. Alternatively, tissue sample holder (40) may be configured to hold any other suitable number of tissue samples. It should be understood that, as with other components described herein, tissue sample holder (40) may be varied, modified, substituted, or supplemented in a variety of ways; and that tissue sample holder (40) may have a variety of alternative features, components, configurations, and functionalities. For instance, tissue sample holder (40) may be alternatively configured such that it has a plurality of discrete tissue sample compartments that may be selectively indexed to cutter lumen (52). Such indexing may be provided automatically or manually. By way of example only, tissue sample holder (40) may be configured and operable in accordance with the teachings of U.S. Pub. No. 2008/0195066, entitled “Revolving Tissue Sample Holder for Biopsy Device,” published Aug. 14, 2008, the disclosure of which is incorporated by reference herein; U.S. Non-Provisional patent application Ser. No. 12/337,997, entitled “Tissue Biopsy Device with Rotatably Linked Thumbwheel and Tissue Sample Holder,” filed Dec. 18, 2008; U.S. Non-Provisional patent application Ser. No. 12/337,911, entitled “Biopsy Device with Discrete Tissue Chambers,” filed Dec. 18, 2008, the disclosure of which is incorporated by reference herein; or U.S. Non-Provisional patent application Ser. No. 12/337,874, entitled “Mechanical Tissue Sample Holder Indexing Device,” filed Dec. 18, 2008, the disclosure of which is incorporated by reference herein. In some other versions, tissue sample holder (40) is configured in accordance with the teachings of U.S. Non-Provisional patent application Ser. No. 12/483,305, entitled “Tetherless Biopsy Device with Reusable Portion,” filed Jun. 12, 2009, the disclosure of which is incorporated by reference herein. Other suitable alternative versions, features, components, configurations, and functionalities of tissue sample holder (40) will be apparent to those of ordinary skill in the art in view of the teachings herein. Alternatively, tissue sample holder (40) may simply be omitted, if desired.

Exemplary Cutter

As shown in FIGS. 2-8, cutter (50) of the present example is tubular and substantially hollow, such that cutter (50) defines a cutter lumen (52). Cutter (50) also has a substantially sharp distal edge (51), such that cutter (50) is operable to sever a biopsy sample from tissue protruding through lateral aperture (24) of needle (20). Alternatively, the distal end of cutter (50) may have any other suitable configuration. In the present example, cutter lumen (52) is in fluid communication with the hollow interior of outer cup (44), such that a vacuum induced within outer cup (44) (e.g., by a vacuum pump within body (30) and/or by a vacuum pump exterior to body (30), etc.) may be communicated from outer cup (44) to cutter lumen (52); and such that tissue samples severed by cutter (50) may be communicated proximally through cutter lumen (52) into outer cup (44). For instance, in some versions, a proximal portion of cutter (50) extends into tissue sample holder (40). In some such versions, a seal (not shown) is provided at the interface of cutter (50) and tissue sample holder (40). Such a seal may be configured to substantially seal the interface of cutter (50) and tissue sample holder (40), even as cutter (50) rotates and translates relative to outer cup (44). Furthermore, cutter (50) is configured such that it remains in sealed fluid communication with the interior of tissue sample holder (40) even when cutter (50) is in a distal-most position. For instance, the length of cutter (50) may be such that at least a portion of cutter (50) is always disposed in outer cup (44) of tissue sample holder (40) during operation of biopsy device (10). Of course, cutter (50) may have any other suitable alternative features or configurations. Similarly, cutter (50) may have any other suitable alternative relationships with tissue sample holder (40).

It should be understood that, as with other components described herein, cutter (50) may be varied, modified, substituted, or supplemented in a variety of ways; and that cutter (50) may have a variety of alternative features, components, configurations, and functionalities. Suitable alternative versions, features, components, configurations, and functionalities of cutter (50) will be apparent to those of ordinary skill in the art in view of the teachings herein.

Exemplary Cutter Actuation Mechanism

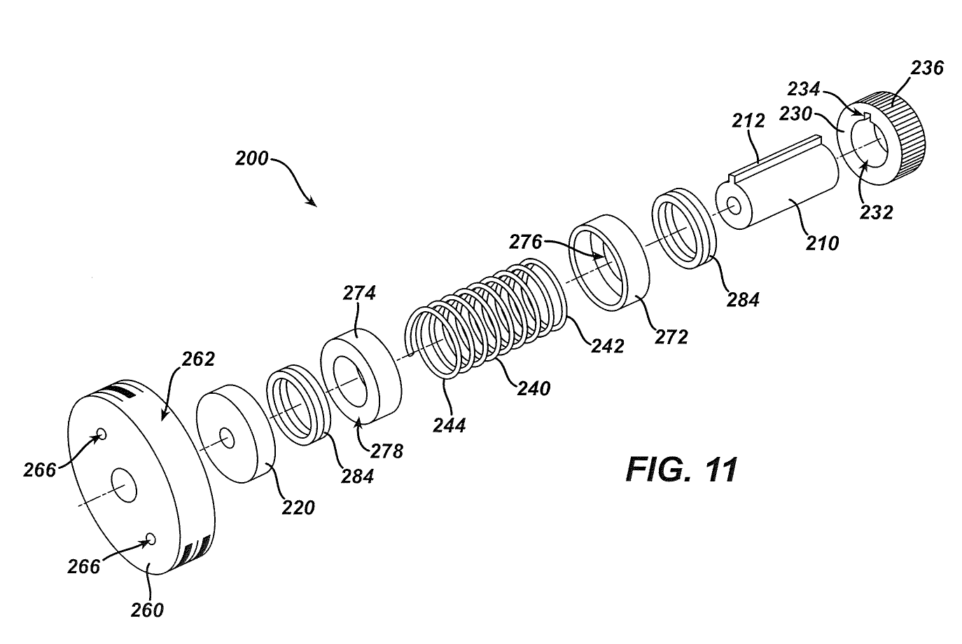

As shown in FIGS. 2, 4, 7, and 9-11, cutter actuation mechanism (200) of the present example comprises a variety of components that interact to provide simultaneous rotation and distal translation of cutter (50) relative to body (30) and needle (20) in a firing stroke. Cutter actuation mechanism (200) is also operable to retract cutter (50) proximally to ready cutter (50) for firing. Such operation of cutter actuation mechanism (200) will be described in greater detail below. While FIG. 11 shows several components of cutter actuation mechanism (200), it should be understood that split nut (250) and cutter (50) are omitted from FIG. 11. It should also be understood that all of the components of cutter actuation mechanism (200) shown in FIG. 11 are positioned coaxially with cutter (50) along part of the length of cutter (50) in the present example.

Cutter actuation mechanism (200) of the present example includes a cutter rotation overmold (210) and a cutter translation overmold (220). In this example, cutter (50) is formed of metal, and each overmold (210, 220) is formed of plastic that is overmolded about the exterior of cutter (50). Each overmold (210, 220) thus translates and rotates unitarily with cutter (50) in this example. Of course, cutter (50) and overmolds (210, 220) may be formed of a variety of other materials (including combinations of materials), may be secured together in any other suitable fashion, and may have any other suitable relationship. Cutter rotation overmold (210) has a substantially cylindraceous shape and includes an external key (212) in the present example. Cutter translation overmold (220) has a disk shape in the present example. It should be understood, however, that overmolds (210, 220) may alternatively have a variety of other types of shapes, features, and configurations.

A cutter rotation gear (230) is positioned about the exterior of cutter rotation overmold (210). In particular, cutter rotation overmold (210) is positioned within an opening (232) defined by cutter rotation gear (230). Opening (232) includes a keyway (234) that complements key (212) of cutter rotation overmold (210). Accordingly, cutter rotation gear (230) and cutter rotation overmold (210) rotate concomitantly in the present example. With cutter rotation overmold (210) being unitarily secured to cutter (50), it should be understood that rotation of cutter rotation gear (230) will rotate cutter (50) in the present example. Cutter rotation gear (230) also has external teeth (236) that are configured to mesh with complementary teeth of drive gear (114), such that drive gear (114) may be rotated to rotate cutter (50), as will be described in greater detail below.

While cutter rotation gear (230) and cutter rotation overmold (210) rotate concomitantly in the present example, cutter rotation overmold (210) is able to translate relative to cutter rotation gear (230). Thus, cutter (50) is able to translate longitudinally relative to cutter rotation gear (230). The longitudinal position of cutter rotation gear (230) remains substantially fixed as cutter (50) translates in the present example. Various suitable ways in which such longitudinal fixation of cutter rotation gear (230) may be provided, while still permitting rotation of cutter rotation gear (230), will be apparent to those of ordinary skill in the art in view of the teachings herein. While just one key (212) and just one keyway (234) are provided in the present example, it should be understood that any other suitable number of keys (212) and keyways (234) may be provided. It should also be understood that cutter rotation gear (230) and cutter rotation overmold (210) may have a variety of alternative complementary features/configurations, in addition to or in lieu of key (212) and keyway (234). For instance, cutter rotation overmold (210) may present an external profile shape (e.g., triangular, square, hexagonal, etc.), with opening (232) of cutter rotation gear (230) having a complementary shape. Other suitable configurations of and relationships between cutter rotation gear (230) and cutter rotation overmold (210) will be apparent to those of ordinary skill in the art in view of the teachings herein.

Cutter actuation mechanism (200) of the present example further includes a spring (240), which is selectively compressed by interaction between an external split nut (250) and an internal spring compression nut (260). Split nut (250) includes a distal internal threaded portion (252) and a proximal internal splined portion (254). The exterior of spring compression nut (260) includes threading that meshes with the threading of threaded portion (252). Thus, as split nut (250) is rotated, spring compression nut (260) translates within distal threaded portion (252). A pair of stabilization arms (264) extend proximally within housing (12) and are disposed through openings (266) formed in spring compression nut (260). Arms (264) substantially stabilize spring compression nut (260) as spring compression nut (260) translates; and further substantially prevent spring compression nut (260) from rotating. In some other versions, however, spring compression nut (260) also rotates. For instance, spring compression nut (260) and split nut (250) may rotate at different speeds, providing a rotation speed differential, which would result in translation of spring compression nut (260). Rotation of split nut (250) is provided through engagement between teeth of drive gear (114) and splines of splined portion (254). In the present example, and as will be described in greater detail below, split nut (250) is only rotated to provide proximal translation of spring compression nut (260); and split nut (250) is not rotated during distal translation of spring compression nut (260), due at least in part to segments (250a, 250b) of split nut (250) being in a separated position.

Spring (240) of the present example comprises a conventional coil spring, and is resiliently biased to assume an extended position. It should be understood, however, that a variety of other types of springs may be used. It should also be understood that a variety of other types of resilient members may be used. In the present example, the proximal end (242) of spring (240) is nested in a proximal collar (272); while the distal end (244) of spring (240) is nested in a distal collar (274). In particular, proximal end (242) of spring (240) bears against a transverse portion (276) of proximal collar (272); while distal end (244) of spring (240) bears against a transverse portion (278) of distal collar (274). A proximal thrust bearing (282) is positioned between proximal collar (272) and the distal face of cutter rotation gear (230). Proximal thrust bearing (282) transfers the longitudinal load imposed by spring (240) on cutter rotation gear (230); yet permits cutter rotation gear (230) to rotate without binding up spring (240). With the longitudinal load of spring (240) being transferred to cutter rotation gear (230), and with cutter rotation gear (230) being longitudinally fixed within body (30) in the present example, it should be understood that proximal end (242) of spring (240) is longitudinally grounded within body (30) by cutter rotation gear (230) in the present example. Of course, spring (240) may be grounded in any other suitable fashion.

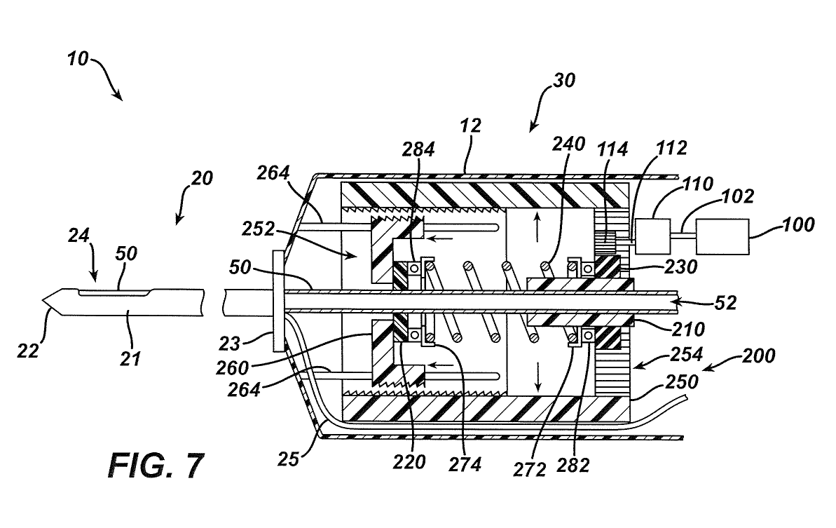

A distal thrust bearing (284) is positioned between distal collar (274) and cutter translation overmold (220). Distal thrust bearing (282) transfers the longitudinal load imposed by spring (240) on cutter translation overmold (220); yet permits cutter translation overmold (220) (and, hence, cutter (50)) to rotate without binding up spring (240). Thus, when spring (240) is compressed and is then allowed to expand distally to its “at rest” length as shown in FIG. 7, spring (240) translates cutter translation overmold (220) and cutter (50) distally. Such distal translation of cutter (50) will be described in greater detail below.

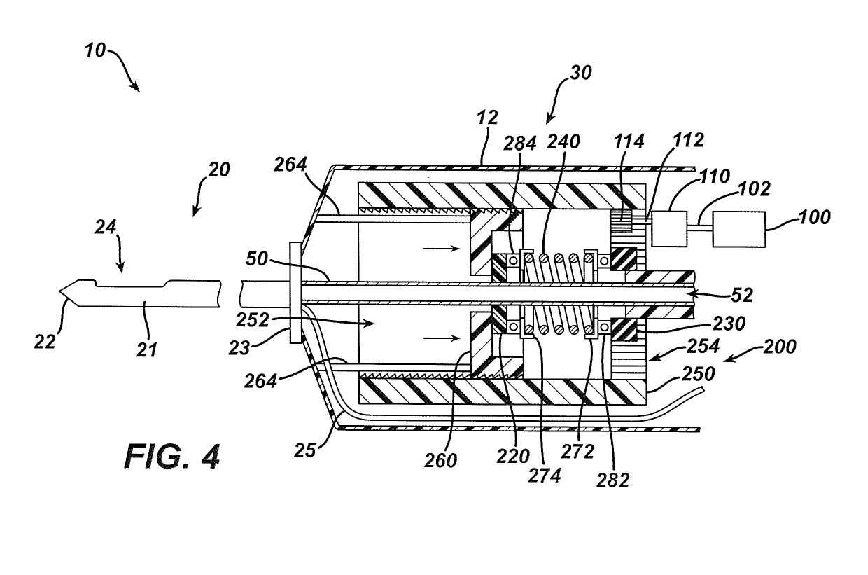

Spring compression nut (260) is configured to push proximally against cutter translation overmold (220) as spring compression nut (260) is translated proximally by rotating split nut (250). While not shown in the present drawings, it should be understood that a thrust bearing or other component may be positioned between spring compression nut (260) and cutter translation overmold (220). With distal end (244) of spring (240) being coupled with cutter translation overmold (220) as described above, and with proximal end (242) of spring (240) being grounded relative to housing (12) by cutter rotation gear (230) as described above, it should be understood that proximal movement of spring compression nut (260) will compress spring (240) in the present example This proximal movement of spring compression nut (260) and resulting compression of spring (240) can be seen by viewing FIGS. 2 and 4 in succession. In particular, FIG. 2 shows spring (240) at an “at rest” length (though spring (240) may actually be loaded to some degree at this position) and spring compression nut (260) at a distal position. FIG. 4 shows spring compression nut (260) translated proximally to a proximal position, thereby compressing spring (240) to load spring (240) (i.e., such that compression spring (240) stores potential energy). As noted above, this proximal translation of spring compression nut (260) is provided by rotating split nut (250), with such rotation being converted to translation of spring compression nut (260) due to interaction between external threading (262) of spring compression nut (260) and internal threaded portion (252) of split nut (250). It should be understood that, once spring compression nut (260) is no longer translating proximally (e.g., rotation of split nut (250) ceases) and spring compression nut (260) remains at the proximal position shown in FIG. 4, spring compression nut (260) holds spring (240) in the compressed and loaded state due to engagement between external threading (262) of spring compression nut (260) and internal threaded portion (252) of split nut (250).

Alternatively, a locking mechanism may selectively engage spring compression nut (260) to selectively restrain the longitudinal position of spring compression nut (260) once spring compression nut (260) has reached the proximal position shown in FIG. 4. Such a locking mechanism may continue to hold spring compression nut (260) in this proximal position even after segments (250a, 250b) of split nut (250) have been separated to disengage spring compression nut (260) as described below. Such a locking mechanism may be lever operated and/or spring operated. Such a locking mechanism may also release spring compression nut (260) in a variety of ways. For instance, such releasing of spring compression nut (260) by the locking mechanism may be provided manually, such that the locking mechanism disengages spring compression nut (260) in response to a user input. Alternatively, releasing of spring compression nut (260) by the locking mechanism may be provided automatically. For instance, the locking mechanism may interact with a feature (e.g., cam and/or lever, etc.) associated with cutter (50), such that the locking mechanism automatically releases spring compression nut (260) when cutter (50) starts rotating for distal translation. Various suitable components, features, configurations, and operabilities of such an optional locking mechanism will be apparent to those of ordinary skill in the art in view of the teachings herein.

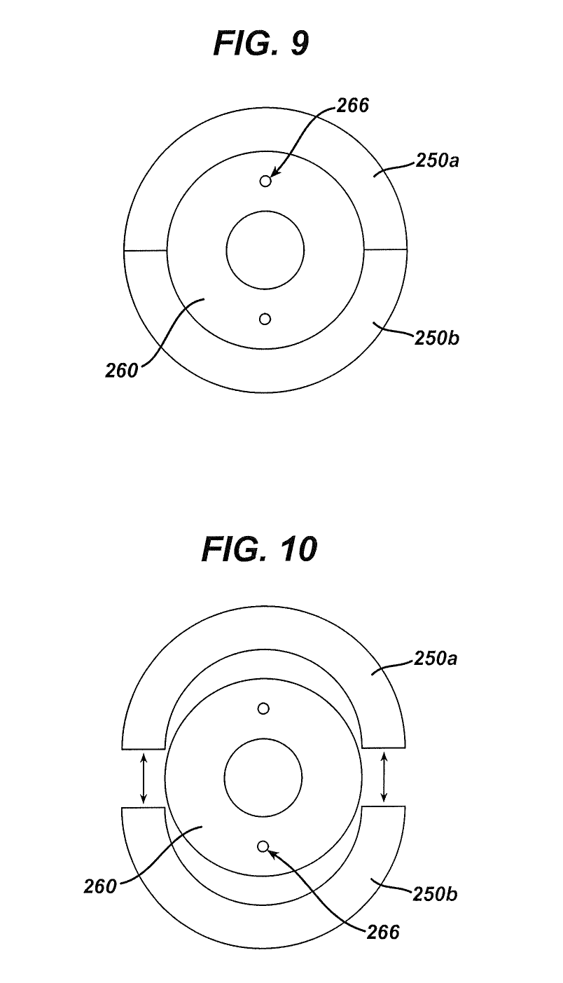

Once spring (240) has been compressed and loaded as shown in FIG. 4, and the user of biopsy device (10) is ready to translate cutter (50) distally in order to sever a biopsy sample from tissue protruding through lateral aperture (24), split nut (250) may separate to release spring compression nut (260). In particular, and as can be seen in FIGS. 9-10, split nut (250) of the present example is formed of two segments (250a, 250b) that are separable by moving segments (250a, 250b) in opposite directions that are along an axis that is transverse to the longitudinal axis of cutter actuation mechanism (200). Such separation of segments (250a, 250b) disengages external threading (262) of spring compression nut (260) from internal threaded portion (252) of split nut (250). With the threading being disengaged, spring compression nut (260) no longer resists the distal bias of the compressed spring (240). As shown in FIG. 7, this results in distal translation of cutter (50) in the present example. When the spring-loaded cutter (50) is fired distally in this fashion (e.g., loaded with potential energy of compressed spring (240)), cutter translation overmold (220) and spring compression nut (260) translate distally with cutter (50). During this distal translation, spring compression nut (260) translates along stabilization arms (264), such that stabilization arms (264) substantially guide and stabilize spring compression nut (260). It should be understood from the teachings herein that, as cutter (50) translates distally, it severs tissue protruding through lateral aperture (24), with such cutting action being achieved through rotation of cutter (50) by motor (100); and through translation of cutter (50) by compressed spring (240). When cutter (50) is retracted proximally for another cutting stroke, such retraction of cutter (50) compresses spring (240) to store potential energy in spring (240) for the next distal translation of cutter (50).

It should be understood that segments (250a, 250b) of split nut (250) may be selectively separated in a variety of ways. For instance, segments (250a, 250b) may be coupled with a mechanism that is mechanically triggered by cutter rotation overmold (210) reaching a proximal position. Such a mechanism may include a cam, a cone, and/or some other feature that is operable to separate segments (250a, 250b). As another merely illustrative example, one or more proximity sensors, other types of sensors, encoders, or other types of devices may be used to monitor the longitudinal position of cutter (50) to trigger separation of segments (250a, 250b) via one or more solenoids or other types of devices. Various other suitable ways in which segments (250a, 250b) may be separated, as well as other suitable ways in which such separation may be triggered, will be apparent to those of ordinary skill in the art in view of the teachings herein. It should also be understood that, while split nut (250) is formed of two segments (250a, 250b) in the present example, split nut (250) may instead be formed of any other suitable number of segments.

As noted above, drive gear (114) is operable to selectively engage cutter rotation gear (230) and splined portion (254) of split nut (250). In the present example, gearbox (110) is operable to selectively shift the position of output shaft (112) to selectively position drive gear (114) into engagement with either cutter rotation gear (230) or splined portion (254) of split nut (250). For instance, FIGS. 2 and 4 show output shaft (112) in a first position, to provide engagement of drive gear (114) with splined portion (254) of split nut (250) during proximal retraction of cutter (50). Thus, in the present example, cutter (50) is not rotated as cutter (50) is retracted proximally. FIG. 7 shows output shaft (112) in a second position, to provide engagement of drive gear (114) with cutter rotation gear (230). Thus, in the present example, cutter (50) is rotated as cutter (50) is fired distally. Various suitable ways in which output shaft (112) may be selectively moved between these first and second positions will be apparent to those of ordinary skill in the art in view of the teachings herein.

In some other versions, gearbox (110) includes a pair of output shafts and a pair of corresponding drive gears. For instance, one such drive gear may be engaged with cutter rotation gear (230) while the other drive gear is engaged with splined portion (254) of split nut (250). In some such versions, gearbox (110) may be operable to selectively activate just one of the output shafts at a time. For instance, the drive gear that is engaged with cutter rotation gear (230) may be deactivated (e.g., such that this drive gear “freewheels”) while the drive gear that is engaged with splined portion (254) of split nut (250) is activated. Similarly, the drive gear that is engaged with splined portion (254) of split nut (250) may be deactivated (e.g., such that this drive gear “freewheels”) while the drive gear that is engaged with cutter rotation gear (230) is activated. Various suitable ways in which gearbox (110) may selectively activate and/or selectively de-activate output shafts will be apparent to those of ordinary skill in the art in view of the teachings herein.

As yet another merely illustrative example, gearbox (110) may include just one output shaft (112) to drive cutter actuation mechanism (200), but output shaft (112) may be positioned (and drive gear (114) may be sized) such that drive gear (114) engages cutter rotation gear (230) and splined portion (254) of split nut (250) simultaneously during proximal retraction of cutter (50). Thus, cutter (50) may rotate as cutter (50) is retracted proximally. When distal firing of cutter (50) is desired, segments (250a, 250b) of split nut (250) may separate to disengage both spring compression nut (260) and drive gear (114) from split nut.

In still another merely illustrative variation, split nut (250) may be configured such that split nut (250) does not rotate at all within body (30), such that split nut (250) lacks splined portion (254), and such that drive gear (114) does not at all engage split nut (250). In some such versions, cutter translation nut (260) is unitary with cutter (50), such that cutter translation nut (260) rotates and translates unitarily with cutter (50). It should be understood that in some such versions, cutter translation nut (260) may be essentially merged with or substitute cutter translation overmold (220). It should also be understood that stabilization arms (264) are omitted in this example, such that cutter translation nut (260) is permitted to rotate within body (30). In this example, drive gear (114) is in constant engagement with teeth (236) of cutter rotation gear (230). Thus, when cutter (50) is in an initial distal position, motor (100) may be activated to rotate drive gear (114), which will in turn rotate cutter (50) and cutter translation nut (260). With split nut (250) being rotationally stationary, the rotation of cutter translation nut (260) relative to split nut (250) provides proximal translation of cutter translation nut (260) and cutter (50), thereby compressing/loading spring (240). Segments (250a, 250b) of split nut (250) may then be separated, disengaging cutter translation nut (260) from split nut (250). Such disengagement allows the distal bias of compressed spring (240) to translate cutter translation nut (260) and cutter (50) distally to the fired, distal position shown in FIG. 7. Drive gear (114) may continue to rotate cutter (50) during this distal translation of cutter (50), as cutter rotation overmold (210) slides relative to cutter rotation gear (230).

It should be understood that cutter translation overmold (220), cutter rotation overmold (210), and/or cutter rotation gear (230) may be formed such that cutter translation overmold (220), cutter rotation overmold (210), and/or cutter rotation gear (230) is/are relatively heavy to obtain a flywheel effect during rotation of cutter (50). Such a flywheel effect may reduce the likelihood of cutter (50) binding or bending while distal edge (51) of cutter (50) cuts through relatively dense or otherwise tough tissue.

In the present example, longitudinal distance traversed by cutter (50) during the proximal stroke (sequence from FIGS. 2-3 to FIGS. 4-6) is equal to the longitudinal distance traversed by cutter (50) during the distal stroke (sequence from FIGS. 4 and 6 to FIGS. 7-8). This distance is approximately 20 mm in the present example. Alternatively, cutter (50) may traverse any other suitable longitudinal distance during a proximal stroke and/or during a distal stroke. Also in the present example, cutter actuation mechanism (200) is configured such that cutter (50) will rotate through approximately 20 revolutions about its longitudinal axis during the distal stroke. Of course, cutter (50) may alternatively rotate through any other suitable number of revolutions about its longitudinal axis during the distal stroke. Various suitable rotation speeds for cutter (50) will also be apparent to those of ordinary skill in the art in view of the teachings herein.

Of course, the above described components, features, configurations, and operabilities of cutter actuation mechanism (200) are merely exemplary. It should be understood that cutter actuation mechanism (200) may be varied or modified in numerous other ways. Various other suitable components, features, configurations, and operabilities of cutter actuation mechanism (200) will be apparent to those of ordinary skill in the art in view of the teachings herein.

Exemplary Method of Operation

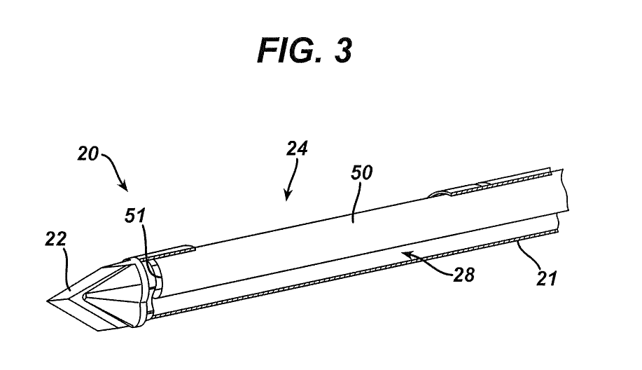

In a merely exemplary use of biopsy device (10), a user first inserts tissue piercing tip (22) into the breast of a patient. During such insertion, cutter (50) may be advanced to the distal-most position, such that lateral aperture (24) of needle (20) is closed as shown in FIGS. 2-3. As also noted herein, such insertion may be performed under visual guidance, stereotactic guidance, ultrasound guidance, MRI guidance, PEM guidance, BSGI guidance, palpatory guidance, some other type of guidance, or otherwise. With needle (20) sufficiently inserted into the patient's breast, the user may then activate motor (100), which may in turn activate cutter actuation mechanism (200). In addition, a vacuum may be induced in tissue sample holder (40) and cutter lumen (52), as well as second lumen (28), as described above. Activation of cutter actuation mechanism (200) may also cause split nut (250) to rotate to retract cutter (50) proximally and compress spring (240), as shown in FIGS. 4-6. As cutter (50) starts retracting and when cutter (50) reaches the retracted position, vacuum communicated through cutter lumen (52) and second lumen (28) may draw tissue into lateral aperture (24) of needle (20). Alternatively, second lumen (28) may be vented at this stage. Once cutter (50) has reached the fully retracted proximal position, lateral aperture (24) is fully open with tissue prolapsed therein.

Segments (250a, 250b) are then separated and drive gear (114) is moved into engagement with cutter rotation gear (230), with drive gear (114) rotating cutter rotation gear (230). This action causes cutter (50) to simultaneously rotate and translate distally. In particular, the distal bias of spring (240) causes cutter (50) to translate distally while motor (100) causes cutter (50) to rotate. It should be understood that segments (250a, 250b) may be separated at approximately the same time as drive gear (114) is moved into engagement with cutter rotation gear (230). Alternatively, driver gear (114) may first be moved into engagement with cutter rotation gear (230), right before segments (250a, 250b) are separated to disengage cutter translation nut (260). Of course, any other suitable relationship between the act of separating segments (250a, 250b) and the act of moving drive hear (114) into engagement with cutter rotation gear (230) may be provided.

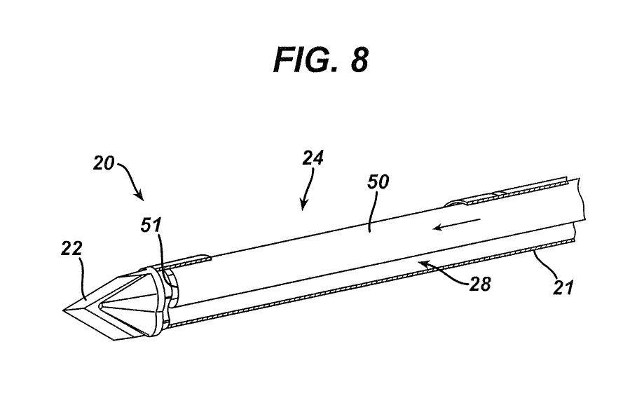

As cutter (50) advances distally, vacuum is still being communicated through vacuum lumen (52), helping to hold prolapsed tissue in place as sharp distal edge (51) of cutter (50) begins to sever the tissue. During this stage, a vacuum may also be communicated to second lumen (28). Alternatively, second lumen (28) may be substantially sealed at this stage. As yet another merely illustrative variation, second lumen (28) may be vented at this stage. Cutter (50) eventually reaches the distal-most position, as shown in FIGS. 7-8, thereby “closing” lateral aperture (24), such that sharp distal edge (51) of cutter (50) completely severs the prolapsed tissue. Vacuum is still being communicated through cutter lumen (52) at this time.

With cutter (50) having reached the distal-most position, the severed tissue sample is initially disposed within cutter lumen (52). At this stage, second lumen (28) may be vented to provide a pressure differential for proximal transport of the severed tissue sample through cutter lumen (52). In particular, the proximal face of the severed tissue sample may be under a vacuum while the distal face of the severed tissue sample may be at atmospheric pressure, which may urge the severed tissue sample proximally through cutter lumen (52) and eventually into outer cup (44) of tissue sample holder (40). In some other versions, saline is communicated through second lumen (28) at this stage instead of atmospheric air. Such saline may also be at atmospheric pressure or may be pressurized. As yet another merely illustrative variation, pressurized air may be communicated through second lumen at this stage.

With cutter (50) having reached the distal-most position and with the severed tissue sample being deposited in tissue sample holder (40), a cutting/sampling stroke/cycle will be complete. The user may then retrieve the severed tissue sample from tissue sample holder (40) for analysis. It should be noted that such cutting/sampling strokes/cycles may be initiated as many times as desired to acquire additional tissue samples. In particular, segments (250a, 250b) of split nut (250) may be re-joined and drive gear (114) may be moved back into engagement with splined portion (254) of split nut (250) to return cutter actuation mechanism (200) to the configuration shown in FIG. 2. Additional cutting/sampling strokes/cycles may be performed several without having to retrieve severed tissue samples from tissue sample holder (40) between each stroke/cycle. Alternatively, the user may wish to at least initially inspect tissue samples, if not remove tissue samples from tissue sample holder (40), between strokes/cycles. It should also be understood that several cutting/sampling strokes/cycles may be performed to acquire several tissue samples without the user having to withdraw needle (20) from the patient's breast. The user may adjust the orientation of lateral aperture (24) about the axis defined by needle (20) by rotating the entire biopsy device (10) between cutting strokes for multiple sample acquisition. Alternatively, biopsy device (10) may be configured such that needle (20) is rotatable relative to body (30), such that needle (20) may be rotated via a thumbwheel or other feature. Once the desired number of tissue samples have been obtained, the user may withdraw needle (20) from the patient's breast. The user may then remove cap (42) from cup (44) and retrieve the tissue samples from the filter tray.

While not shown in the drawings, it should be understood that biopsy device (10) may include one or more switches to selectively activate cutter actuation mechanism (200). For instance, in some versions biopsy device (10) includes a single externally manipulatable switch that is operable to trigger a full biopsy sampling cycle. Thus, when a user activates such a switch, the same may activate motor (100) to rotate split nut (250) to retract cutter (50) and thus load spring (240); and segments (250a, 250b) may be automatically separated to allow spring (240) to fire cutter (50) distally once cutter (50) has reached a sufficiently proximal position. Such a single switch may require continuous pressing/activation by the user to complete the biopsy sampling cycle, such that cutter actuation mechanism (200) will cease motion when the user releases the single button. In some such versions, the cutter actuation mechanism (200) may still automatically cease motion once a biopsy sampling cycle has been completed, even if the user continues to press/activate the switch. Biopsy device (10) may thus require the user to release then re-press/re-activate the switch in order to initiate a second biopsy sampling cycle. In another variation, a single switch may require just an initial act of pressing/activation by the user to complete a full biopsy sampling cycle, such that the user need not continue to press/activate the switch throughout the biopsy sampling cycle. As yet another merely illustrative variation, biopsy device (10) may include a forward/reverse switch, such that a first interaction by the user is required in order to translate cutter (50) proximally; and such that a second interaction by the user is required in order to translate cutter (50) distally. Various other suitable ways in which biopsy device (10) may provide user interaction to activate cutter actuation mechanism (200) will be apparent to those of ordinary skill in the art in view of the teachings herein. Similarly, various suitable circuitry components and configurations that may be used to provide control of cutter actuation mechanism (200) will be apparent to those of ordinary skill in the art in view of the teachings herein.

It should be understood that any of a variety of operations may occur at the end of a cutting stroke. For instance, biopsy device (10) may provide a variety of forms of feedback to inform the user that a cutting stroke as been completed. By way of example only, biopsy device (10) may provide an electronic beep or other audible indication, a mechanical audible indication (e.g., a loud click), a visual indication (e.g., a light illuminating or flashing), or some other type of audible and/or visual indication. Alternatively, and particularly in versions where cup (44) is transparent, the user may know that a cutting stroke is complete by simply watching tissue sample holder (40) until the user sees a tissue sample being deposited on the filter tray. Still other suitable ways in which biopsy device (10) may operate at the end of a cutting stroke and/or initiate a subsequent cutting stroke will be apparent to those of ordinary skill in the art in view of the teachings herein.

In versions of biopsy device (10) where an electronic based audible and/or visual indication of the end of a cutting stroke is provided, as well as versions of biopsy device (10) where a control module automatically deactivates motor (100) or disengages a clutch or provides some other type of automated response, there are a variety of ways in which the end of a cutting stroke and/or some other stage of cutter (50) actuation (e.g., cutter (50) reaching proximal-most position, etc.) may be sensed. For instance, a portion of cutter (50) may include a magnet, and a hall effect sensor may be positioned in body (30) to sense the presence of the magnet when cutter (50) reaches the distal-most position at the end of a cutting stroke and/or the proximal-most position at the end of a retraction stroke. As another merely illustrative example, an encoder wheel may be coupled with cutter (50) or a rotating component of cutter actuation mechanism (200), such that the longitudinal position of cutter (50) may be determined based on a number of rotations. Other suitable ways in which one or more stages of a cutting cycle may be sensed (e.g., electronically, mechanically, electro-mechanically, manually, etc.) will be apparent to those of ordinary skill in the art in view of the teachings herein.

Of course, the above examples of use of biopsy device (10) are merely illustrative. Other suitable ways in which biopsy device (10) may be used will be apparent to those of ordinary skill in the art in view of the teachings herein.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Embodiments of the present invention have application in conventional endoscopic and open surgical instrumentation as well as application in robotic-assisted surgery.

Embodiments of the devices disclosed herein can be designed to be disposed of after a single use, or they can be designed to be used multiple times. Embodiments may, in either or both cases, be reconditioned for reuse after at least one use. Reconditioning may include any combination of the steps of disassembly of the device, followed by cleaning or replacement of particular pieces, and subsequent reassembly. In particular, embodiments of the device may be disassembled, and any number of the particular pieces or parts of the device may be selectively replaced or removed in any combination. Upon cleaning and/or replacement of particular parts, embodiments of the device may be reassembled for subsequent use either at a reconditioning facility, or by a surgical team immediately prior to a surgical procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, embodiments described herein may be processed before surgery. First, a new or used instrument may be obtained and if necessary cleaned. The instrument may then be sterilized. In one sterilization technique, the instrument is placed in a closed and sealed container, such as a plastic or TYVEK bag. The container and instrument may then be placed in a field of radiation that can penetrate the container, such as gamma radiation, x-rays, or high-energy electrons. The radiation may kill bacteria on the instrument and in the container. The sterilized instrument may then be stored in the sterile container. The sealed container may keep the instrument sterile until it is opened in a medical facility. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometries, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

What is claimed is:

1. A biopsy device, comprising:
(a) a body;
(b) a needle extending distally from the body, wherein the needle has a tip and a lateral aperture proximal to the tip;
(c) a cutter slidably disposed within the needle, wherein the cutter is movable from a proximal position to a distal position to sever tissue protruding through the lateral aperture;
(d) a resilient member configured to resiliently bias the cutter toward the distal position; and
(e) a motor operable to move the cutter from the distal position to the proximal position against the resilient bias of the resilient member.

2. The biopsy device of claim 1, wherein the motor comprises an electric motor.

3. The biopsy device of claim 1, wherein the motor is further operable to rotate the cutter.

4. The biopsy device of claim 3, further comprising:
(a) a first cutter rotation member unitarily secured to the cutter; and
(b) a second cutter rotation member engaged with the first cutter rotation member, wherein the second cutter rotation member is configured to communicate rotation from the motor to the first cutter rotation member to rotate the cutter, wherein the longitudinal position of the second cutter rotation member is substantially fixed relative to the body;
wherein the first cutter rotation member slidably disposed within the second cutter rotation member such that the first cutter rotation member is configured to translate relative to the second cutter rotation member.

5. The biopsy device of claim 1, further comprising:
(a) a first nut; and
(b) a second nut disposed within the first nut, wherein the second nut is movable proximally to compress the resilient member, wherein the motor is operable to rotate the first nut to translate the second nut proximally.

6. The biopsy device of claim 5, wherein the first nut comprises a split nut formed by at least two segments, wherein the split nut is engaged with the second nut when the at least two segments are in a first position, wherein the split nut is disengaged from the second nut when the at least two segments are in a second position.

7. The biopsy device of claim 6, wherein the at least two segments are substantially adjacent to each other when the at least two segments are in the first position, wherein the at least two segments are substantially separated from each other when the at least two segments are in the second position.

8. The biopsy device of claim 1, wherein the resilient member comprises a coil spring.

9. The biopsy device of claim 9, wherein the coil spring is disposed coaxially about the cutter.

10. The biopsy device of claim 10, wherein the coil spring has a distal end and a proximal end, the biopsy device further comprising:
(a) a first bearing coupled with the distal end of the coil spring; and
(b) a second bearing coupled with the proximal end of the coil spring;
wherein the cutter is rotatable;
wherein the first and second bearings are configured to substantially prevent the coil spring from rotating as the cutter is rotated.

11. The biopsy device of claim 1, further comprising:
(a) a first rotatable member, wherein the motor is operable to selectively rotate the first rotatable member to move the cutter from the distal position to the proximal position; and
(b) a second rotatable member, wherein the motor is operable to selectively rotate the second rotatable member to rotate the cutter.

12. The biopsy device of claim 11, further comprising a drive shaft in communication with the motor, wherein the drive shaft is selectively movable between a first position and a second position, wherein the drive shaft is engaged with the first rotatable member when the drive shaft is in the first position, wherein the drive shaft is engaged with the second rotatable member when the drive shaft is in the second position.

13. The biopsy device of claim 12, further comprising a gearbox coupled with the motor, wherein the drive shaft forms an output of the gearbox.

14. The biopsy device of claim 1, wherein the cutter is rotatable within the needle, the biopsy device further comprising:
(a) a cutter rotation member unitarily secured to the cutter, wherein the motor is operable to selectively rotate the cutter rotation member to rotate the cutter; and
(b) a cutter translation member unitarily secured to the cutter, wherein the resilient member is engaged with the cutter translation member to resiliently bias the cutter toward the distal position.

15. The biopsy device of claim 14, wherein the cutter translation member is positioned distal to the cutter rotation member.

16. The biopsy device of claim 14, wherein the cutter is formed of metal, wherein the cutter rotation member is formed of plastic overmolded about the cutter, wherein the cutter translation member is formed of plastic overmolded about the cutter.

17. The biopsy device of claim 1, wherein the motor is positioned within the body.

18. The biopsy device of claim 1, further comprising a tissue sample holder coupled with the body, wherein the cutter is configured to communicate severed tissue samples to the tissue sample holder.

19. A biopsy device, comprising:
(a) a body;
(b) a needle extending distally from the body, wherein the needle has a tip and a lateral aperture proximal to the tip;
(c) a cutter slidably disposed within the needle, wherein the cutter is movable from a proximal position to a distal position to sever tissue protruding through the lateral aperture;
(d) a resilient member configured to resiliently bias the cutter toward the distal position;
(e) a split nut, wherein the split nut comprises a plurality of segments movable between a first position and a second position; and
(f) a cutter retraction member engaged with the resilient member, wherein the cutter retraction member is further engaged with the split nut when the plurality of segments are in the first position;
wherein the split nut and the cutter retraction member are operable to move the cutter from the distal position to the proximal position against the resilient bias of the resilient member when the plurality of segments are in the first position;
wherein the split nut is configured to release the cutter retraction member when the plurality of segments are in the second position, thereby allowing the resilient member to translate the cutter distally.

20. A biopsy device, comprising:
(a) a body;
(b) a needle extending distally from the body, wherein the needle has a tip and a lateral aperture proximal to the tip;
(c) a cutter slidably disposed within the needle, wherein the cutter is movable from a proximal position to a distal position to sever tissue protruding through the lateral aperture;
(d) a resilient member configured to resiliently bias the cutter toward the distal position; and
(e) a motor operable to move the cutter from the distal position to the proximal position against the resilient bias of the resilient member, wherein the motor is further operable to rotate the cutter as the cutter is translated distally by the resilient member.
